SEQUENCE LISTING (6 sequences)

SEQ ID NO: 1 - DNA, 19 bp, Artificial Sequence
  Bacterial tuf forward primer
ccgtgcagaagctggtaga

SEQ ID NO: 2 - DNA, 22 bp, Artificial Sequence
  Bacterial tuf Reverse Primer
gagatcgagaacacgtcttcga

SEQ ID NO: 3 - DNA, 24 bp, Artificial Sequence
  Bacterial tuf Probe
  misc_feature: 5` FAM
  misc_feature: 3` Phosphate
  misc_feature (9)..(10): BHQ-2 Quencher
ttccggagccggttcgtgccatcg

SEQ ID NO: 4 - DNA, 19 bp, Artificial Sequence
  Human beta-globin Forward Primer
gccagtgccagaagagcca

SEQ ID NO: 5 - DNA, 25 bp, Artificial Sequence
  Human beta-globin Reverse Primer
tccttaaacctgtcttgtaaccttg

SEQ ID NO: 6 - DNA, 36 bp, Artificial Sequence
  Human beta-globin Probe
  misc_feature: 5` CY5.5
  misc_feature: 3` Phosphate
  misc_feature (18)..(19): BHQ-2 Quencher
cctccctgctcctgggagtagattggccaaccctag

(19) United States
(12) Patent Application Publication
(10) Pub. No.: US 2024/0084404 A1
(43) Pub. Date: Mar. 14, 2024

(54) SELECTIVE LYSIS OF HUMAN BLOOD CELLS

(71) Applicant: Roche Molecular Systems, Inc., Pleasanton, CA (US)

(72) Inventors: Rajiv Dua, Manteca, CA (US); Slav Dugenny, San Leandro, CA (US); Xiaoyun Zhang, San Jose, CA (US)

(21) Appl. No.: 18/269,171

(22) PCT Filed: Dec. 23, 2021
(86) PCT No.: PCT/EP2021/087443

§ 371 Date: Jun. 22, 2023

Related U.S. Application Data
(60) Provisional application No. 63/130,625, filed on Dec. 25, 2020.

(51) Int. Cl. C12Q 1/689; C12Q 1/6844

(52) U.S. Cl. CPC C12Q 1/689; C12Q 1/6844; C12Q 1/6806

(57) ABSTRACT

The present invention relates to methods for the specific lysis of eukaryotic cells in a sample containing microorganisms such as bacterium and fungus by incubating the sample in a buffer of around pH 9 that contains a non-ionic detergent.

### BACKGROUND OF THE INVENTION

Sepsis due to blood stream infections (BSI) is a life-threatening condition with increased rate of hospitalizations. Appropriate and timely detection of sepsis is critical because delay in diagnosis results in multi-organ failure and increased mortality. The polymerase chain reaction (PCR) provides a rapid way to detect bacterial pathogens in sepsis induced by BSI. One of the most important factor in PCR-based sepsis diagnostics is to reliably amplify and detect low levels of nucleic acid copies arising from pathogens in the blood. This requires PCR to achieve high detection sensitivity.

Molecular diagnostics aims at the rapid detection of minute amounts of pathogens (typically bacteria) in samples such as blood. Blood is however a complex matrix and comprises white blood cells (leukocytes) for the adaptive immune system, red blood cells (erythrocytes) for oxygen transport, and platelets (thrombocytes) for wound healing. This complicates the direct detection of pathogens in samples such as whole blood, which contain a high amount of cellular material.

For PCR based methods the amount of bacteria in a fresh blood sample is theoretically high enough to be detected without further cultivation of the bacteria present within such sample. However, to allow an early detection of minute amounts of bacteria, large volumes of blood are required. The high amount of DNA in especially white blood cells dramatically increases the background in DNA based detection methods. Also the presence of heme from hemoglobin strongly decreases the activity of DNA polymerase. A microliter of human blood contains about 4,000 to 11,000 white blood cells and about 150,000 to 400,000 platelets. The concentration of DNA in blood is between 30 and 60 μg/ml. It is extremely challenging to detect in a volume of 10 ml of whole blood the presence of about 10 to 100,000 of a bacterial species.

However, abundance of human DNA, arising from the presence of white-blood cells and other DNA fragments floating in the blood, hampers PCR-based detection sensitivity for sepsis diagnostics. Apart from interfering with the PCR reaction itself the amount of mammalian DNA increases the viscosity of a sample. In addition, proteins and membranes from the lysed mammalian cells form complexes which prevent the filtration of a sample. This is particularly a problem for miniaturized devices. Further dilution of the, already large sample volume, results in unacceptable long manipulation steps.

Molysis™ (from the company Molzym GmbH & Co. KG, Bremen, Germany) uses chaotropic agents and detergents to lyse selectively mammalian cells. This lysis step is followed by a digest with a DNase, which is not affected by this chaotropic agent/detergent. Alternative approaches such as commercialized by Roche Diagnostics (LightCycler® Septifast™) rely on PCR primer pairs, which are specifically designed to prevent non-specific binding to human DNA and amplification of human DNA. However, there is a need in the art for a pre-analytic buffer that depletes the human DNA without compromising the nucleic acid detection from bacterial pathogens.

### SUMMARY OF THE INVENTION

Methods as described in the present invention allow a selective lysis of white and red blood cells in a sample while bacteria and fungi remain intact and unlysed (either dead or alive).

Particular and preferred aspects of the invention are set out in the accompanying independent and dependent claims. Features from the dependent claims may be combined with features of the independent claims and with features of other dependent claims as appropriate.

One aspect of the invention relates to a method for the selective lysis of eukaryotic cells, in particular animal cells, within a sample containing or suspected to contain a microorganism. This method comprises the steps of providing a sample with eukaryotic cells, in particular animal cells, containing or suspected to contain a microorganism, adding a non-ionic detergent and a buffer to the sample to obtain a solution with a pH of about 9.0 or slightly lower, wherein the ratio between the volume of added detergent and added buffer and the volume of sample is between 2:1 and 1:10, and incubating the solution for a time period sufficiently long to lyse the eukaryotic cells, in particular animal cells.

In some embodiments, the non-ionic detergent is present in a concentration within the range of between 0.1 and 5% (w/v % or v/v %). In certain embodiments, the non-ionic detergent is present in a concentration within the range of between 0.1 and 2% (w/v % or v/v %), particularly in a concentration within the range of between 0.1 and 1% (w/v % or v/v %). In particular embodiments, the non-ionic detergent is present in a concentration of around 1% (w/v % or v/v %).

In some embodiments, the non-ionic detergent is polidocanol. In some embodiments, the non-ionic detergent is polidocanol at a concentration of between 0.1 and 2% (v/v %), %), particularly at a concentration of between 0.1 and 1% (v/v %). In some embodiments, the non-ionic detergent is polidocanol at a concentration of around 1% (v/v %).

In some embodiments, the time period sufficiently long to lyse the eukaryotic cells, in particular animal cells is between 30 seconds and 10 minutes, more specifically between 1 and 8 minutes, more specifically between 2 and 6 minutes.

In some embodiments, lysis can be performed at a temperature of between 15° C. and 30° C., more specifically, between 18° C. and 27° C., more specifically, between 20° C. and 25° C. In some embodiments, the lysis can be performed around or at room temperature.

In some embodiments, the animal cells are mammalian cells.

In some embodiments, the sample is a mammalian blood sample. In certain embodiments, the mammalian blood sample is a whole blood sample. In particular embodiments, the mammalian blood sample is a plasma sample or a platelet preparation.

In some embodiments, the microorganism is a bacterium or fungus.

In certain embodiments, the ratio between the volume of the added detergent and buffer and the volume of sample is between 2:1 and 1:5. In other embodiments, the ratio between the volume of the added detergent and buffer and the volume of sample is between 1:1 and 1:10. In yet other embodiments, the ratio between the volume of the added detergent and buffer and the volume of sample is between 1:1 and 1:5.

In some embodiments, the final solution will have a pH of about 8.5 to 9.0. In certain embodiments, the final solution will have a pH of about 8.7 to 9.0. In particular embodiments, the final solution will have a pH of about 8.8 to 9.0. In specific embodiments, the final solution will have a pH of about 8.9 to 9.0.

In some embodiments, the buffer is an alkaline buffer. Herein the alkaline buffer may have a pKa value of about 9.0. In certain embodiments, the buffer is sodium carbonate. In certain embodiments, the buffer has sufficient buffer capacity that when mixed with the sample in ratios according to the present invention, the pH of the final solution is around 9.0 or slightly lower. In particular embodiments, the alkaline buffer has a pKa of around 9.0 so the final solution will have a pH of about 9.0 or slightly lower, in particular that the final solution will have a pH of about 8.5 to 9.0; more specifically, of about 8.7 to 9.0; more specifically, of about 8.8 to 9.0; more specifically, of about 8.9 to 9.0.

In certain embodiments, the method may further comprise a “neutralization step”. In some embodiments, the step comprises adding after the selective lysis according to the disclosure an acid or acidic buffer to obtain a pH between about 7 and 9. In some embodiments, the step of neutralizing the buffer comprises adding an acid or acidic buffer to obtain a pH of around 7.0.

In certain embodiments, the methods as described above are followed by a step of lysing the microorganisms. In certain embodiments, the methods as described above are followed by detection of the microorganisms by performing a polymerase chain reaction (PCR) assay.

### DETAILED DESCRIPTION OF THE INVENTION

“Blood cells” in the context of the present invention relates to mammalian cells present in blood and includes red blood cells (erythrocytes), white blood cells (leukocytes) and blood platelets (thrombocytes).

“Whole blood” in the context of the present invention relates to unprocessed blood comprising blood plasma and cells, potentially treated with an anti-coagulant.

“Sample” relates to an aqueous suspension comprising cellular material and comprises body fluids such as lymph, cerebrospinal fluid, blood (whole blood and plasma), saliva, but also comprises, e.g., the aqueous fraction of homogenized suspensions such as, e.g., muscles, brain, liver, or other tissues.

“Eukaryotic” in the present invention relates to any type of eukaryotic organism excluding fungi, such as animals, in particular animals containing blood, and comprises invertebrate animals such as crustaceans and vertebrates. Vertebrates comprise both cold-blooded (fish, reptiles, amphibians) and warm blooded animal (birds and mammals). Mammals comprise in particular primates and more particularly humans.

“Selective lysis” as used in the present invention is obtained when in a sample (such as blood) the percentage of micro-organism cells (such as bacterial cells) in that sample that remain intact is significantly higher (e.g. 2, 5, 10, 20, 50, 100, 250, 500, or 1000 time more) compared to the percentage of the eukaryotic cells from the organism from which the sample is collected that remain intact.

“Microorganism” as used in the present invention relates to bacteria (gram-positive and gram-negative bacteria, as well as bacterial spores) and unicellular fungi such as yeast and molds, which are present in the organism from which a sample has been collected, typically as a pathogen.

A first aspect of the present invention relates to a method for the selective lysis of eukaryotic cells, in particular animal cells, within a sample, which contains or is suspected to contain, microorganisms such as bacteria. The aim of the method is to increase the sensitivity of a test for the detection of minute amounts of bacteria in a sample (i.e. less than 10000, 1000, 100 or even less micro-organisms per ml of sample). As explained in the background of the invention, DNA from eukaryotic cells, in particular from animal cells, in a sample interferes with PCR based detection methods and this DNA, together with proteins and membranes form aggregates, which increase viscosity after lysis and which has a dramatic impact on the filtration of a lysed sample. To solve this problem, the eukaryotic cells, in particular animal cells, are selectively lysed, whereby a substantial part (i.e., more than 20%, 40%, 60%, 80%, 90% or even more that 95%) of the microorganisms remains alive, or if killed by the treatment, still comprise the bacterial DNA within the cell wall. In methods as described in the present invention the above mentioned problems are addressed.

Methods as described in the present invention are particularly applicable to any type of sample wherein the detection of DNA from microorganisms, particularly from bacteria, is impaired by the presence of other cells comprising DNA, in particular cells from a host wherein the micro-organism is present as a pathogen.

Methods as described in the present invention are now further illustrated for embodiments wherein the presence of minute amounts of bacteria in a mammalian blood sample is investigated. The blood sample can be stored as whole blood or a processed fraction such as plasma or a platelet preparation. Typically, methods as described in the present invention are performed on freshly isolated whole blood. Such samples are generally treated with, e.g., heparin, EDTA or citrate to avoid coagulation.

Alternatively, the method is performed on fresh blood by collecting the blood from the vein directly in a tube with detergent and buffer.

Accordingly, a fresh blood sample or a preserved sample is supplemented with a buffer and a non-ionic detergent. The selection of the buffer and its concentration are chosen in order to compensate the buffering capacity of the blood sample provided and to obtain a pH of around 9.0, or slightly lower, in particular a pH of about 8.5 to 9.0. In particular, the buffer and its concentration are chosen to obtain a pH of about 8.7 to 9.0; more specifically, of about 8.8 to 9.0; more specifically, of about 8.9 to 9.0. Equally, the buffer is sufficiently concentrated such that at most a buffer volume of 200%, 150%, 100%, 50%, 20% or 10% of the sample volume is added to the sample to obtain the required change in pH. In particular, the buffer comprises a detergent and is added at a ratio of the volume of the added detergent and buffer and the volume of sample of between 2:1 and 1:10. Further, the ratio between the volume of the added detergent and buffer and the volume of sample may be between 2:1 and 1:5, between 1:1 and 1:10 or between 1:1 and 1:5.

Suitable buffers in the context of the present invention typically have a pKa around 9.0, and include a carbonate buffer with an optimal buffering capacity in the above mentioned pH ranges. Suitable detergents are non-ionic detergents, which at the one hand have a lytic effect on the eukaryotic cells, in particular animal cells, only and on the other hand have a solubilizing effect on DNA and proteins. In a particular embodiment, the non-ionic detergent is polidocanol, also referred as polydocanol. Polidocanol relates to a chemical compound or composition consisting of a mixture of polyethylene glycol monododecyl ethers averaging about 9 ethylene oxide groups per molecule. It could be described by the molecular formula (C₂H₄O)ₙC₁₂H₂₆O or HO(CH₂CH₂O)ₙ(CH₂)₁₁CH₃ whereby n is about or exactly 9, i.e. the median number of ethylene glycol moieties is about or exactly 9 as a result of the production method wherein lauryl alcohol is reacted with ethylene oxide (ethoxylation). Use of polidocanol for purification of nucleic acids is disclosed in U.S. Pat. No. 8,192,958, which is incorporated herein by reference in its entirety.

The most effective concentration of detergent depends from detergent to detergent, but typically is within the range of between 0.1 and 5%, particularly between 0.1 and 2%, more particularly between 0.1 and 1%. Depending from the detergent (solid or liquid) % refers to respectively w/v % or v/v %. In one particular embodiment, the buffer for selective lysis of human blood cells is 1000 mM Sodium carbonate (Na₂CO₃)/1% polidocanol, pH 9.0.

The incubation of a blood sample in the presence of buffer and detergent is performed within 10 minutes, preferably between 30 seconds and 10 minutes and more preferably between about 1 to 3, 1-5, 1-8, 2-6 or 1-10 minutes, at temperatures between 10 and 30° C., more preferably around room temperature.

Methods according to the present invention have the advantage that a selective lysis is obtained below 10 minutes, at temperatures below 30° C. Accordingly, the methods can be generally performed at ambient temperatures without the need to heat the sample.

Optionally, after the lysis the pH of the lysed sample is brought to a neutral value (i.e., pH of around 7.0) by the addition of an acid or acidic buffer in a neutralization step. It was found that a lysed sample at neutral pH could be stored for a prolonged time (up to 1, 2, 6, 12 or even 24 hours) without further lysis of bacterial cells and without dramatic changes in the fluidic properties of the lysed sample.

Generally, methods in accordance with the present invention comprise a step wherein the intact bacterial cells are separated from the sample, typically performed by centrifugation or filtration.

It is to be understood that although preferred embodiments, specific constructions and configurations, as well as materials, have been discussed herein for devices according to the present invention, various changes or modifications in form and detail may be made without departing from the scope and spirit of this invention.

### EXAMPLES

The present invention describe a novel buffer that depletes human cells and DNA without compromising the nucleic acid detection from bacterial pathogens. The formulation of the buffer is 1000 mM Na₂CO₃/1% polidocanol, pH 9.0. In the existing literature, carbonate/non-ionic detergent buffers with pH 9.5 and above showed selective lysis of human cells to deplete human DNA. Surprisingly, the present invention depletes human DNA at pH 9.0. Compared to the carbonate/non-ionic detergents that works at pH 9.5 and above, the present invention has an advantage to protocols that specifically use RNA-based PCR detection for sepsis. RNA is specifically prone to degradation under basic pH conditions. Thus, as compared to carbonate/non-ionic detergents with pH 9.5 and above, lower pH of 9.0 in the present buffer system helps to minimize the risk of RNA degradation. This trend is observed when comparing the performance of 1000 mM Na₂CO₃/1% polidocanol, pH 9.0 buffer with 1000 mM Na₂CO₃/1% polidocanol, pH 9.6 buffer (Tables 2 and 3). In addition, a higher stability of carbonate salts in pH 9.0 buffer can be observed.

### Example 1: Preanalytics to Remove Human DNA

The Pseudomonas aeruginosa bacterial strain was grown in LB medium to log phase and an appropriate volume equivalent to 1000 CFU/mL was spiked into the freshly collected human whole blood. Blood containing the spiked bacteria was combined with the selected buffers (Table 2) at 1:1 ratio, mixed, and kept at room temperature for 10 minutes to allow fragmentation of human chromatin. After the incubation step, an equal volume of neutralization buffer (1M Tris-HCL, pH 4.5) was added and the sample was centrifuged at 3220 g for 15 minutes. The supernatant was discarded and the cell pellet was resuspended in 1 mL of Cobas® PCR media (4.2M Guanidine HCL, 50 mM Tris-HCL, pH 7.5) containing 1% β-mercaptoethanol. The sample was transferred to a MagNa Lyser instrument (Roche Diagnostics) for cell lysis that was conducted at the maximum speed for 70 seconds. The lysed sample was loaded on the Cobas® 6800 System and total nucleic acid were isolated in 50 lit of elution buffer using a standard Cobas® assay workflow.

### Example 2: Reverse-Transcription PCR Assay

Assay contain 2 primers and 1 probe for the Pseudomonas aeruginosa specific tuf target and 2 primers and 1 probes for the human β-globin target. The sequences of the primers and probes are shown in Table 1. The reaction was run in the Lightcycler® 480 instrument (Roche Diagnostics) using a temperature profile, which consisted of a uracil-DNA N-glycosylase incubation step (50° C. for 120 s, 94° C. for 5 s), a pre-PCR step (one cycle: 55° C. for 120 s, 60° C. for 360 s, 65° C. for 240 s), five cycles of 95° C. for 5 s and 55° C. for 30 s (1^{st} measurement at the end of each cycle) followed by 45 cycles of 91° C. for 5 s and 58° C. for 25 s (2^{nd} measurement at the end of each cycle) and finished with a cooling step (40° C. for 120 s). The real-time PCR assay mixture consisted of 15 μL of Cobas® PCR generic Master mix containing 100-300 nM of primers and 50-200 nM of probes, 10 μL of Mn²⁺ at 3 mM, and 25 lit of total nucleic acid template. Cycle threshold (Ct) values for the PCR assay were calculated using a custom optimized Algorithm Testing Framework (ATF) software

TABLE 1
Primer and probe sequences

| | | SEQ |
|---|---|---|
| | | ID |
| | Sequence (5′ to 3′) | NO: |
| Bacterial tuf | | |
| Forward primer | CCGTGCAGAAGCTGGTAGA | 1 |
| Reverse primer | GAGATCGAGAACACGTCTTCGA | 2 |
| Probe | <FAM_Thr>TTCCGGAGC<BHQ_2> | 3 |
| | CGGTTCGTGCCATCG<Phos> | |
| Human β-globin | | |
| Forward primer | GCCAGTGCCAGAAGAGCCA | 4 |
| Reverse primer | TCCTTAAACCTGTCTTGTAACCTTG | 5 |
| Probe | <CY5.5>CCTCCCTGCTCCTGGGAG | 6 |
| | <BHQ_2>TAGATTGGCCAACCCTAG | |
| | <Phos> | |

<FAM_Thr> and <CY5.5> are the reporter dyes; <BHQ2> is the Black Hole Quencher-2; <Phos> is Phosphate.

### Example 3: Experimental Results

TABLE 2
Ct values of PCR assay using bacterial tuf gene and human
beta globin gene in different pre-analytics buffer

| | Bacterial tuf | | | Human β-globin | | |
|---|---|---|---|---|---|---|
| | Ct | | | Ct | | |
| | Replicate 1 | Replicate 2 | Average | Replicate 1 | Replicate 2 | Average |
| No pre-analytic buffer | 27.9 | 28.6 | 28.2 | 22.6 | 22.3 | 22.5 |
| control | | | | | | |
| Carbonate/polidocanol | 27.9 | 28.6 | 28.2 | 24.7 | 24.8 | 24.7 |
| buffer, pH 9.6 | | | | | | |
| Carbonate/polidocanol | 26.5 | 26.7 | 26.6 | 24.5 | 24.9 | 24.7 |
| buffer, pH 9.0 | | | | | | |

TABLE 3
Change in Ct values compared to “No pre-analytic buffer control”

| Buffer | Bacterial tuf | Human β-globin |
|---|---|---|
| Carbonate/polidocanol | 0.00 | 2.3 |
| buffer, pH 9.6 | | |
| Carbonate/polidocanol | −1.61 | 2.3 |
| buffer, pH 9.0 | | |

1. A method for the selective lysis of eukaryotic cells within a sample containing or suspected to contain a microorganism, said method comprising the steps of:
a) providing a sample with eukaryotic cells containing or suspected to contain a microorganism,
b) adding a non-ionic detergent and a buffer to said sample to obtain a solution with a pH of around 9.0 or slightly lower, wherein the ratio between the volume of added detergent and buffer and the volume of sample is between 2:1 and 1:10,
c) incubating said solution for a time period sufficiently long to lyse the eukaryotic cells.

2. The method of claim 1, wherein the non-ionic detergent is present in a concentration within the range of between 0.1 and 5% (w/v % or v/v %).

3. The method of claim 2, wherein the non-ionic detergent is present in a concentration of around 1% v/v %.

4. The method of any one of claims 1 to 3, wherein the non-ionic detergent is polidocanol.

5. The method of any one of claims 1 to 4, wherein the eukaryotic cells are animal cells.

6. The method of claim 5, wherein the animal cells are mammalian cells.

7. The method of any one of claims 1 to 4, wherein the sample is a mammalian blood sample.

8. The method of any one of claims 1 to 7, wherein the microorganism is a bacterium or fungus.

9. The method of any one of claims 1 to 8, wherein the buffer is sodium carbonate.

10. The method of any one of claims 1 to 9 further comprising: d) neutralizing the buffer with an acid to obtain a pH of around 7.0.

11. The method of any one of claims 1 to 10 further comprising: e) lysing said microorganisms.

12. The method of any one of claims 1 to 11 further comprising: f) detecting the microorganisms by performing a polymerase chain reaction (PCR) assay.
